(19) United States
(12) Patent Application Publication
(10) Pub. No.: US 2026/0166290 A1
(43) Pub. Date: Jun. 18, 2026

(54) ORAL MICRONEEDLE PATCH

(71) Applicant: LTS Lohmann Therapie-Systeme AG, Andernach (DE)

(72) Inventors: Markus MÜLLER, Troisdorf (DE); Anna SCHLÜTER, Wachtberg (DE); Michael LINN, Waldböckelheim (DE)

(21) Appl. No.: 18/713,419

(22) PCT Filed: Nov. 25, 2022
(86) PCT No.: PCT/EP2022/083371

§ 371 Date: May 24, 2024

(30) Foreign Application Priority Data
Nov. 25, 2021 (DE) 10 2021 130 954.9

(51) Int. Cl. A61M 37/00

(52) U.S. Cl. CPC A61M 37/0015; A61M 2037/0023; A61M 2037/0046; A61M 2037/0061

(57) ABSTRACT

The invention relates to an oral thin film comprising a matrix layer containing at least one polymer, wherein a microneedle system is applied to at least one surface of the oral thin film, a method for preparing such an oral thin film, a composite comprising such a thin film and an application aid, which comprises at least one polymer, for the oral thin film, wherein the oral thin film and the application aid are firmly joined to one another, a method for preparing the oral thin film, a method for preparing the composite, and the oral thin film and the composite for use as a medicinal product.

The invention relates to an oral thin film, a method for preparing such an oral thin film, a composite comprising said oral thin film and an application aid, a method for preparing said composite, and said oral thin film or said composite for use as a medicinal product.

Oral thin films are thin films, usually containing an active pharmaceutical ingredient, which are placed directly in the oral cavity or applied to the oral mucosa, where they dissolve. In particular, they are thin polymer-based films containing active ingredients which, when applied to a mucous membrane, especially the oral mucosa, release the active ingredient directly into it. These oral thin films are generally not tacky on the outside. The excellent blood circulation of the oral mucosa ensures that the active ingredient is quickly absorbed into the bloodstream. This dosage system has the advantage that the active ingredient is largely absorbed through the mucous membrane, thus avoiding the “first-pass metabolism” that occurs with the conventional dosage form of an active ingredient in tablet form, which is usually taken with liquid, which can be disadvantageous. The active ingredient can be dissolved, emulsified, or dispersed in the film. Suitable active ingredients can also be swallowed after dissolving the oral thin film in the mouth and thus be absorbed via the gastrointestinal tract.

However, there is a large number of active pharmaceutical ingredients that cannot be applied via the oral mucosa due to their size or other properties, such as hydrophobicity. The oral mucosa forms an insurmountable barrier for them.

The present invention therefore was based on the problem of providing an oral thin film which overcomes these disadvantages and with which active ingredients, for which this route of administration was previously not accessible, can be applied via the oral mucosa as well.

This problem was solved by an oral thin film according to claim 1. Such an oral thin film comprises a matrix layer containing at least one polymer and is characterized in that a microneedle system is applied to at least one surface of said oral thin film, wherein the microneedle system is applied to the at least one surface of said oral thin film by means of an adhesive layer.

The microneedles can penetrate the oral mucosa and thus reduce its resistance so that an active pharmaceutical ingredient can penetrate it more easily.

The application of oral thin films can be problematic. Due to the shape, size, properties and intended application site of the oral thin film, but also due to the user's impairments, difficulties may arise in applying the oral thin film to the desired site. Particularly in the case of oral thin films, which are equipped with microneedles on one surface to increase the active ingredient flux, it is important that these microneedles are not damaged before any application.

These disadvantages can be eliminated by an application aid for the oral thin film.

Conventional application aids can improve this, but they usually have to be laboriously removed from the oral thin film and/or have a complicated structure. There is also a risk that the user may inadvertently swallow the application aid.

The present application therefore also was based on the problem of providing an application aid for the oral thin film which overcomes the above disadvantages. The application aid should have a high haptic stability in order to apply the oral thin film at the desired location. In addition, the risk of swallowing the application aid as a whole should be minimized, and the risk of the user destroying or damaging the oral thin film before its application should be minimized.

This problem is surprisingly solved by a composite according to claim 9, i.e. by a composite comprising the oral thin film according to the invention and an application aid, which comprises at least one polymer, for the oral thin film, which is characterized in that the oral thin film and the application aid are firmly joined to each other.

The advantage of such a joint is that the user does not have to touch the oral thin film itself in order to apply it. The contact is preferably made exclusively via the application aid. Furthermore, in a preferred embodiment, the application aid can be designed in such a way that it dissolves in the oral cavity of the user. Due to the firm joint with the application aid, oral thin films that are equipped with microneedles on one surface can be applied advantageously.

All features of one embodiment can be combined with features of another embodiment if the features of the different embodiments are not incompatible.

The terminology used in the description of the present disclosure is intended only to describe certain embodiments and should not be construed as limiting the subject matter. As used in the present description and claims, the singular forms “a”, “an” are to be understood to include the plural forms as well, unless the context clearly dictates otherwise. This also applies vice versa, i.e., the plural forms also include the singular forms. It is also understood that the term “and/or” as used herein refers to and includes all possible combinations of one or more of the associated listed elements. Moreover, it is to be understood that the terms “include”, “including”, “comprise”, and/or “comprising”, when used in the present description and claims, specify the presence of the specified features, steps, elements, and/or components, but do not exclude the presence or addition of one or more other features, steps, elements, components, and/or groups thereof.

In the present description and claims, the terms “include”, “comprise”, and/or “comprising” may also mean “consisting of”, i.e., the presence or addition of one or more other features, steps, elements, components, and/or groups is excluded.

The oral thin film according to the invention comprises a matrix layer containing at least one polymer, wherein a microneedle system is applied to at least one surface of said oral thin film.

In a preferred embodiment, the oral thin film according to the invention consists of the matrix layer and the microneedle system.

A microneedle system, also known as a microneedle array, preferably comprises a system comprising a plurality of microneedles on a support. The support preferably comprises at least one polymer.

The needles preferably have a length of 5 μm to 1000 μm.

The density of the needles is preferably from 50 to 1000, in particular from 100 to 600 needles per cm².

The microneedle system is preferably applied to at least one, preferably exactly one, surface of the oral thin film.

The surface to which the microneedle system is applied is preferably the largest surface of the oral thin film.

In one embodiment, the microneedle system is larger than the oral thin film.

In one embodiment, the microneedle system is smaller than the oral thin film.

In one embodiment, the microneedle system and the oral thin film are of approximately the same size.

In a preferred embodiment, the at least one polymer comprises a water-soluble polymer.

Water-soluble polymers comprise natural or synthetic polymers that are chemically quite different and whose common characteristic is their solubility in water or aqueous media. A prerequisite is that these polymers have a sufficient number of hydrophilic groups for water solubility and are not cross-linked. The hydrophilic groups may be non-ionic, anionic, cationic, and/or zwitterionic.

Water-soluble is preferably understood to mean a solubility of more than 100 g/L in water at 25° C.

The at least one water-soluble polymer is preferably selected from the group comprising starch, starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acid, polyacrylates, polyvinylpyrrolidones, vinylpyrrolidone-vinyl acetate copolymers, polyvinyl alcohols, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan, and/or natural gums.

The oral thin film according to the invention is preferably characterized in that the at least one polymer, preferably the water-soluble polymer, is present in the matrix layer in an amount of 10 to 100% (w/w), or 30 to 95% (w/w), preferably 60 to 90% (w/w), particularly preferably 70 to 90% (w/w), based on the total weight of said matrix layer.

In one embodiment, the oral thin film according to the invention is further preferably characterized in that the matrix layer contains at least one active pharmaceutical ingredient.

In one embodiment, the oral thin film according to the invention is further preferably characterized in that the microneedle system comprises at least one active pharmaceutical ingredient.

In one embodiment, the oral thin film according to the invention is further preferably characterized in that the matrix layer and/or the microneedle system contain at least one active pharmaceutical ingredient. In this embodiment, the matrix layer and the microneedle system may contain the same active pharmaceutical ingredient, or they may contain different active pharmaceutical ingredients each.

The amount of the at least one active pharmaceutical ingredient is preferably 1 to 60% (w/w), preferably about 1 to 40% (w/w), based on the total weight of the matrix layer.

The absolute amount of the at least one active pharmaceutical ingredient is preferably from 1 μg to 100 mg, in particular from 1 μg to 10 mg.

The at least one active pharmaceutical ingredient is not limited in principle.

Preferably, the at least one active pharmaceutical ingredient is selected from the group comprising the active ingredient classes of analgesics, hormones, hypnotics, sedatives, antiepileptics, amphetamines, psychoneurotropic agents, neuromuscular-blocking agents, antispasmodics, antihistamines, antiallergics, cardiotonic agents, antiarrhythmics, diuretics, hypotensive agents, vasopressors, antidepressants, antitussives, expectorants, thyroid hormones, sex hormones, antidiabetics, antitumor agents, antibiotics, vaccines, chemotherapeutics and/or narcotics.

Specific examples comprise acetaminophen, adrenaline, alprazolam, amlodipine, anastrozole, apomorphine, aripiprazole, atorvastatin, baclofen, benzocaine, benzocaine/menthol, benzydamine, buprenorphine, buprenorphine/naloxone, buprenorphine/naloxone/cetirizine, cetirizine, chlorpheniramine, clomipramine, dexamethasone, dextromethorphan, dextromethorphan/phenylephrine, diclofenac, diphenhydramine, diphenhydramine/phenylephrine, donepezil, dronabinol, epinephrine, escitalopram, famotidine, fentanyl, glimepiride, GLP-1 peptides, granisetron, insulin, insulin nanoparticles, insulin/GLP-1 nanoparticles, ketamine, ketoprofen, ketotifen, caffeine, levocetirizine, loperamide, loratadine, meclizine, methylphenidate, midazolam, mirodenafil, montelukast, multimeric-001, naloxone, nicotine, nitroglycerin, olanzapine, olopatadine, ondansetron, oxybutynin, pectin, pectin/menthol, pectin/ascorbic acid, pediaSUNAT (artesunate and amodiaquine), piroxicam, phenylephrine, prednisolone, pseudoephedrine, risperidone, rivastigmine, rizatriptan, selegilines, senna glycosides, sildenafil citrate, simethicone, sumatriptan, tadalafil, testosterone, triamcinolone acetonide, triptan, tropicamide, voglibose, zolmitriptan, zolpidem, and/or pharmaceutically acceptable salts of these compounds.

The active pharmaceutical ingredient can also be a mixture of different active ingredients.

In one embodiment, the oral thin film, in particular the matrix layer, is present in the form of a smooth film.

A smooth film is preferably characterized by the fact that it is not in the form of a foam and does not exhibit any significant unevenness when analyzed optically without aids or magnification. A film can be produced, for example, by spreading and drying a polymer-containing solution, emulsion, suspension, or melt.

In another embodiment, the oral thin film, in particular the matrix layer, is present in the form of a solidified foam having cavities.

The cavities and the associated larger surface area of the films facilitate the access of water or saliva or other body fluids into the interior of the dosage form, thus accelerating the dissolution of the dosage form and the release of the active ingredient.

In the case of a rapidly absorbing active ingredient, transmucosal absorption can also be improved due to the rapid dissolution.

On the other hand, the wall thickness of the cavities mentioned is preferably low, as these represent solidified bubbles, for example, so that these cavities dissolve or are destroyed quickly.

A further advantage of this embodiment is that, despite the comparatively high weight per unit area, a faster drying time can be achieved than with a comparable non-foamed composition due to the preparation as a foam.

Preferably, the oral thin film is characterized in that the cavities are isolated from each other and are preferably in the form of bubbles, wherein the cavities are filled with air or a gas, preferably with an inert gas, particularly preferably with nitrogen, carbon dioxide, helium, or a mixture of at least two of these gases.

According to another embodiment, it is provided that the cavities are connected with each other, preferably by forming a continuous channel system penetrating the matrix.

Preferably, the aforementioned cavities have a volume fraction of 5 to 98%, preferably 50 to 80%, based on the total volume of the matrix layer. In this way, the advantageous effect of accelerating the dissolution of the matrix layer is favorably influenced.

Furthermore, surface-active substances or surfactants can be added to the matrix for foam formation or to the resulting foam before or after drying in order to improve the stability of the foam before or after drying.

Another parameter that influences the properties of the dosage form according to the invention is the diameter of the cavities or bubbles. The bubbles or cavities are preferably produced with the aid of a foam whipping machine, with which the diameter of the bubbles can be adjusted over a wide range, almost at will. Thus, the diameter of the bubbles or cavities can range from 0.01 to 350 μm. Particularly preferably, the diameter is in the range of 10 and 200 μm.

The oral thin film or rather the matrix layer according to the invention preferably has a surface area of 0.5 cm² to 10 cm², particularly preferably of 1.5 cm² to 7.5 cm².

The oral thin film or rather the matrix layer according to the invention is preferably characterized in that the weight per unit area of the oral thin film or rather the matrix layer is 10 to 500 g/m², preferably 100 to 400 g/m².

The weight per unit area of the oral thin film or rather the matrix layer is preferably at least 10 g/m², more preferably at least 20 g/m², or at least 30 g/m², or most preferably at least 50 g/m², or is less than or equal to 400 g/m², more preferably less than or equal to 350 g/m², or less than or equal to 300 g/m², or most preferably less than or equal to 150 g/m². Preferably, the weight per unit area of the oral thin film or rather the matrix layer is 10 to 400 g/m², more preferably 20 to 350 g/m², or 30 to 300 g/m², and most preferably 50 to 150 g/m².

Preferably, the oral thin film or the matrix layer has a layer thickness of about 20 μm to about 500 μm, particularly preferably from about 50 μm to about 300 μm.

The microneedle system preferably comprises microneedles with a length of 10 to 1000 μm, preferably with a length of 100 μm to 600 μm, particularly preferably from 250 μm to 350 μm.

Furthermore, the microneedle system is preferably a microneedle system based on starch, starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acids, polyacrylates, polyvinylpyrrolidones, polyvinyl alcohols, poly(lactide-co-glycolide), hyaluronic acid, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan, and/or natural gums, wherein this group is not exhaustive.

Preferably, the microneedles comprise a polymer that is also present in the support.

Preferably, the microneedles comprise a polymer that is also present in the oral thin film, in particular in the matrix layer.

Preferably, the support of the microneedles comprises a polymer that is also present in the oral thin film, in particular in the matrix layer.

Preferably, the microneedles and their support comprise a polymer that is also present in the oral thin film, in particular in the matrix layer.

In specific embodiments, the microneedle system can also consist of materials such as ceramics, steel, polymers, and/or SiO₂.

In a preferred embodiment, the oral thin film and/or the microneedle system further comprise at least one excipient selected from the group comprising colorants, flavors, sweeteners, plasticizers, taste-masking agents, emulsifiers, enhancers, pH regulators, humectants, preservatives, and/or antioxidants.

These excipients are preferably each contained in an amount of 0.1 to 30.0% (w/w) in the oral thin film or in the microneedle system.

In one embodiment, the oral thin film with microneedles can also be produced by using a negative mold of the microneedles during the production of the oral thin film, so that the oral thin film and the microneedles are monolithic.

Alternatively, the microneedle system can be manufactured separately and attached to the oral thin film using any suitable means of attachment.

The microneedle system and oral thin film can be joined, for example, by means of an adhesive layer, preferably a water-soluble adhesive layer and/or by means of a seal, preferably a heat seal.

Sealing comprises, but is not limited to, joining by embossing and/or heat sealing.

Heat sealing refers to the joining of the microneedle system and the oral thin film by punctiform heating and pressing. The punctiform heating softens or melts the at least one polymer that is present in the microneedle system or in the oral thin film, which leads to a firm joint after renewed cooling. Preferably, the temperature is punctiformly raised to a temperature that is above the melting temperature or glass transition temperature of the respective polymer.

The usual temperatures for heat sealing are about 50° C. to 200° C. The heat sealing is preferably carried out for about 5 seconds, more preferably for about 3 seconds, and most preferably for about 2 seconds or less at a temperature of about 50° C. to 200° C.

The microneedle system is applied to the at least one surface of the oral thin film by means of an adhesive layer, in particular a water-soluble adhesive layer.

Water-soluble is to be understood here as defined above.

Water-soluble adhesive layers are particularly suitable as adhesive layers, as described in DE 10 2014 127 452 A1, the relevant content of which is hereby included in full.

Suitable adhesive layers comprise at least one water-soluble polymer and at least one plasticizer, wherein the at least one water-soluble polymer in the at least one water-soluble adhesive layer comprises shellac, a vinylpyrrolidone-vinyl acetate copolymer, a polyvinylcaprolactam-polyvinyl acetate-polyethylene glycol copolymer, hydroxypropyl cellulose or hydroxypropyl methyl cellulose, and/or polyvinylpyrrolidone, and wherein the at least one plasticizer in the at least one water-soluble adhesive layer comprises glycerol, polyethylene glycol, in particular polyethylene glycol 200, and/or tributyl citrate. The weight ratio of the at least one water-soluble polymer to the at least one plasticizer in the at least one adhesive layer is preferably about 85 to 50 to about 15 to 50.

Such an adhesive layer, which contains at least one water-soluble polymer and at least one plasticizer, can firmly adhere two further layers, which are not sticky themselves, to each other as an intermediate water-soluble adhesive layer, and thus enable the formation of multilayer composites.

In principle, the oral thin film is not limited in the number of layers it can contain.

Thus, embodiments are conceivable in which the oral thin film comprises further layers. The above definitions apply analogously to the other layers.

The present invention further relates to a composite comprising an oral thin film as described above and an application aid for the oral thin film.

The application aid comprises at least one polymer and is firmly joined to the oral thin film.

It is to be understood that the application aid is joined to the oral thin film on the surface to which the microneedle system is not applied.

The term “application aid” can be understood in the broadest possible sense here.

Preferably, the application aid comprises a flat structure with a preferred thickness of 0.1 mm to 4 mm, in particular from 0.1 mm to 2 mm, or 0.5 mm to 2 mm or from 100 μm to 500 μm, to the surface of which the oral thin film is firmly attached.

The application aid can take any form. For example, the application aid can be essentially round, oval, or elliptical.

However, the application aid can also be triangular or square, if necessary with rounded corners. Shapes in other possible polygons, if necessary with rounded corners, are also possible.

Mixed forms are possible as well, wherein the application aid has, for example, a first round, oval. or elliptical area and a second area attached to it, which is, for example, strip-shaped, triangular, or square (also polygonal), so that the application aid as a whole has a shape that resembles the shape of a tennis racket, wherein the second area here preferably serves as a “handle” and the first area serves to hold the oral thin film.

In the present case, “firmly joined” means that the application aid and the oral thin film are joined to each other in such a way that the user cannot separate the joint without destroying the film. This joint should also not be impaired over a longer storage period.

The composite according to the invention is preferably characterized in that the application aid comprises a first area and a second area.

The oral thin film is preferably arranged in the first area of the application aid.

The second area of the application aid is preferably designed in such a way that a user can touch the application aid in such a way that the oral thin film is not touched.

Preferably, the oral thin film is attached to a first third or a first half of the surface of the application aid. This leaves two thirds or a second half of the application aid free, which can serve as a “handle” for applying the oral thin film.

The composite according to the invention is preferably characterized in that the application aid has a surface area which is at least twice as large, preferably 2 to 10 times or 2 to 4 times as large, as the surface area of the oral thin film.

In possible embodiments, the application aid has a surface area of 1 cm² to 30 cm², preferably from 2 cm² to 15 cm² or 2 cm² to 10 cm².

In possible embodiments, the oral thin film has a surface area of 0.3 cm² to 10 cm², preferably 0.5 cm² to 7 cm².

In possible embodiments, the application aid has a length of 1 cm to 10 cm, preferably 2 cm to 5 cm.

In possible embodiments, the application aid has a width of 0.5 cm to 5 cm, preferably 1 cm to 3 cm.

In possible embodiments, the oral thin film has a length of 0.5 cm to 5 cm, preferably 1 cm to 4 cm.

In possible embodiments, the oral thin film has a width of 0.5 cm to 4 cm, preferably 1 cm to 4 cm.

In possible embodiments, the application aid has a thickness of 0.1 mm to 4 mm, preferably 0.1 mm to 2 mm, or 100 μm to 500 μm.

In possible embodiments, the oral thin film has a thickness of 0.01 mm to 2 mm, preferably 0.05 mm to 1 mm, or 100 μm to 400 μm.

In possible embodiments, the application aid has a weight per unit area of 20 g/m² to 800 g/m², more particularly 20 g/m² to 500 g/m².

In possible embodiments, the oral thin film has a weight per unit area of 10 g/m² to 600 g/m², preferably 10 g/m² 400 g/m².

The application aid can be formed single-layered or multi-layered.

The composite according to the invention is preferably characterized in that the application aid comprises at least one water-soluble polymer.

Water-soluble polymers comprise natural or synthetic polymers that are chemically quite different and whose common characteristic is their solubility in water or aqueous media. A prerequisite is that these polymers have a sufficient number of hydrophilic groups for water solubility and are not cross-linked. The hydrophilic groups may be non-ionic, anionic, cationic and/or zwitterionic.

Water-soluble is preferably understood to mean a solubility of more than 100 g/L in water at 25° C.

The at least one water-soluble polymer is preferably selected from the group consisting of starch, starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acids, polyacrylates, polyvinylpyrrolidones, vinylpyrrolidone-vinyl acetate copolymers, polyvinyl alcohols, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan and/or natural gums, polyvinyl alcohols being particularly preferred.

The composite according to the invention is preferably characterized in that the at least one polymer, preferably the water-soluble polymer, is present the matrix layer in an amount of 10 to 100% (w/w), or 10 to 95% (w/w), preferably 60 to 90% (w/w), particularly preferably 80 to 90% (w/w), based on the total weight of the application aid.

The composite according to the invention is preferably characterized in that the application aid as a whole is water-soluble. Here too, water-soluble is preferably understood to mean a solubility of more than 100 g/L in water at 25° C.

The composite according to the invention is preferably characterized in that the application aid comprises aromas, taste masking agents, and/or anesthetics or local anesthetics.

As anesthetics or local anesthetics, aminoamides such as lidocaine, amino esters such as procaine, fomocaine, quinisocaine, or cyclonine are suitable here.

These are preferably contained in an amount of 0.1 to 10% (w/w) or 0.1 to 5% (w/w), based on the total weight of the application aid.

The composite according to the invention is preferably characterized in that the application aid further comprises at least one excipient selected from the group comprising colorants, sweeteners, plasticizers, emulsifiers, enhancers, pH regulators, humectants, preservatives and/or antioxidants.

Each of these excipients is preferably present in an amount of 0.1 to 40% (w/w), particularly preferably 0.1 to 30% (w/w), more particularly preferably 0.1 to 15% (w/w), and even more preferably 0.1 to 10% (w/w), or 0.1 to 5% (w/w), based on the total weight of the application aid.

In particular, the stability of the application aid can be adjusted via the content of plasticizer.

Suitable plasticizers include glycerol, for example.

The plasticizer(s) is/are preferably present in an amount of 0.1 to 15% (w/w), most preferably 0.1 to 10% (w/w), or 0.1 to 5% (w/w), particularly about 5% (w/w), based on the total weight of the application aid.

Preferably, the composite according to the invention is characterized in that the application aid is in the form of a film that is as smooth as possible.

In another embodiment, the composite according to the invention is characterized in that the application aid is in the form of a solidified foam having cavities.

The cavities and the associated larger surface area of the application aid particularly facilitate the access of water or saliva or other body fluids into the interior of the application aid, thus accelerating the dissolution of the application aid.

On the other hand, the wall thickness of the cavities mentioned is preferably low, as these represent solidified bubbles, for example, so that these cavities dissolve or are destroyed quickly.

A further advantage of this embodiment is that, despite the comparatively high weight per unit area due to the preparation as a foam, a faster drying time can be achieved than with a comparable non-foamed composition.

Preferably, the composite according to the invention is characterized in that the cavities are isolated from each other and are preferably in the form of bubbles, wherein the cavities are filled with air or a gas, preferably with an inert gas, particularly preferably with nitrogen, carbon dioxide, helium, or a mixture of at least two of these gases.

According to another embodiment, it is provided that the cavities are connected to each other, preferably by forming a continuous channel system penetrating the matrix.

Preferably, the aforementioned cavities have a volume fraction of 5 to 98%, preferably 50 to 80%, based on the total volume of the application aid. In this way, the solution of the application aid is influenced in a favorable way.

Furthermore, surface-active substances or surfactants can be added to the application aids for foam formation or to the resulting foam before or after drying in order to improve the stability of the foam before or after drying.

Another parameter that influences the properties of the application aid according to the invention is the diameter of the cavities or bubbles. The bubbles or cavities are preferably produced with the aid of a foam whipping machine, with which the diameter of the bubbles can be adjusted over a wide range, almost at will. Thus, the diameter of the bubbles or cavities can range from 0.01 to 350 μm. Particularly preferably, the diameter is in the range of 10 and 200 μm.

The composite according to the invention is preferably characterized in that the application aid and the oral thin film are joined with each other each other by means of an adhesive layer, preferably a water-soluble adhesive layer and/or by means of a seal, preferably a heat seal.

An adhesive layer is understood to be a layer that can act as an adhesive, as defined in DIN EN 923:2016-03.

Water-soluble adhesive layers are particularly suitable as adhesive layers, as described in DE 10 2014 127 452 A1, the relevant content of which is hereby included in full.

Suitable adhesive layers comprise at least one water-soluble polymer and at least one plasticizer, wherein the at least one water-soluble polymer in the at least one water-soluble adhesive layer comprises shellac, a vinylpyrrolidone-vinyl acetate copolymer, a polyvinylcaprolactam-polyvinyl acetate-polyethylene glycol copolymer, hydroxypropyl cellulose or hydroxypropyl methyl cellulose, and/or polyvinylpyrrolidone, and wherein the at least one plasticizer in the at least one water-soluble adhesive layer comprises glycerol, polyethylene glycol, in particular polyethylene glycol 200, and/or tributyl citrate. The weight ratio of the at least one water-soluble polymer to the at least one plasticizer in the at least one adhesive layer is preferably about 85 to 50 to about 15 to 50.

Such an adhesive layer, which contains at least one water-soluble polymer and at least one plasticizer, can firmly adhere two further layers, which are not sticky themselves, to each other as an intermediate water-soluble adhesive layer, and thus enable the formation of multilayer composites.

Sealing is understood to mean any possible way in which the application aid and the oral thin film can be joined with each other. However, this does not concludingly comprise joints by embossing and/or heat sealing.

Heat sealing refers to the joining of the application aid and the oral thin film by punctiform heating and pressing. The punctiform heating melts the at least one polymer that is present in the application aid or in the oral thin film, which leads to a firm joint after renewed cooling. Preferably, the temperature is punctiformly raised to a temperature that is above the melting temperature or glass transition temperature of the respective polymer.

The usual temperatures for heat sealing are about 50° C. to 200° C.

The heat sealing is preferably carried out for about 5 seconds, preferably for about 3 seconds, and particularly preferably for about 2 seconds or less at a temperature of about 50° C. to 200° C.

The composite according to the invention is further preferably characterized in that the application aid dissolves in the oral cavity of a patient within 10 s to 5 min, preferably from 30 s to 3 min.

The composite according to the invention is further preferably characterized in that the application aid and the oral thin film dissolve in the oral cavity of a patient within 10 s to 5 min, preferably from 30 s to 2 min.

The present invention also relates to a method for preparing an oral thin film as described above, comprising the steps of
providing an oral thin film comprising at least one polymer and at least one active pharmaceutical ingredient,
providing a microneedle system, and
firmly joining the microneedle system and the oral thin film, wherein firmly joining comprises the following steps
applying an adhesive layer to a surface of the oral thin film,
applying the microneedle system to this adhesive layer, and
drying the adhesive layer to obtain a firm composite.

All the embodiments and definitions described above for the oral thin film, the microneedle system and for the adhesive layer apply analogously to the method for preparing an oral thin film as described above.

It is irrelevant whether the microneedle system or the oral thin film is provided in the first step.

If the oral thin film is present in the form of a foam, the method also comprises the step of foaming the solution, emulsion, melt, or suspension by introducing a gas or gas mixture, by chemical gas generation, or by expansion of a dissolved gas.

In the present case, “firmly joined” preferably means that the microneedle system and the oral thin film are joined to each other in such a way that the user cannot separate the joint without destroying the film. This joint should also not be impaired over a longer storage period.

An alternative method for preparing an oral thin film, in particular as described above, preferably comprises the following steps for forming a firm joint between the microneedle system and the oral thin film:
heating the oral thin film to a temperature that is above the softening temperature of the at least one polymer,
applying the microneedle system to a surface of the heated oral thin film to obtain a loose composite, and
cooling the loose composite to obtain a firm composite.

Preferably, heating comprises heating to a temperature of 50 to 100° C., preferably for about 0.5 to 5 min.

The present invention further relates to an oral thin film obtainable according to the method described above.

The present invention also relates to a method for preparing a composite comprising an oral thin film as described above and an application aid for the oral thin film as described above, comprising the steps of:
providing an application aid,
providing an oral thin film, and
firmly joining the application aid to the oral thin film.

All embodiments defined above for the composite, the oral thin film and the application aid apply analogously to the method according to the invention.

It is irrelevant whether the application aid or the oral thin film is provided in the first step.

Preferably, the application aid and/or the oral thin film is provided by a method comprising preparing a solution, emulsion, melt, or suspension comprising all ingredients of the application aid or the oral thin film, respectively, followed by spreading and drying the solution, emulsion, melt, or suspension.

All suitable shapes can be cut or punched out of these dried solutions, emulsions, melts, or suspensions.

If the application aid and/or the oral thin film is present in the form of a foam, the method also comprises the step of foaming the solution, emulsion, melt, or suspension by introducing a gas or gas mixture, by chemical gas generation, or by expansion of a dissolved gas.

The method for preparing a composite as described above, preferably comprises the following steps for forming a firm joint between the application aid and the oral thin film:
heating the oral thin film and/or the application aid to a temperature that is above the softening temperature of the respective at least one polymer,
applying the oral thin film to a surface of the application aid to obtain a loose composite, and
cooling the loose composite to obtain a firm composite.

Preferably, heating here comprises heating to a temperature of 50 to 100° C., preferably for about 0.5 to 5 min.

An alternative method for preparing a composite as described above, preferably comprises the following steps for forming a firm joint between the application aid and the oral thin film:
applying an adhesive layer to a surface of the oral thin film or to a surface of the application aid,
joining the oral thin film and the application aid via this adhesive layer, and
drying the adhesive layer to obtain a firm composite.

The adhesive layer is to be understood as defined above.

Furthermore, the present invention relates to an oral thin film as described above or obtainable by the method described above as a medicinal product.

Furthermore, the present invention relates to a composite as described above or obtainable by the method described above as a medicinal product.

All embodiments defined above for the composite, the oral thin film and the application aid apply analogously to the medical use according to the invention.

### DESCRIPTION OF THE FIGURES

FIG. 1 shows a possible design of the oral thin film. The film is an oral thin film (3) onto which microneedles (5) are applied. These microneedles (5) can either be applied to the oral thin film with an adhesive layer (4) or attached thereon using alternative attachment measures.

FIG. 2 shows an oral thin film (7) equipped with a microneedle system (8). Here, the oral thin film is larger than the microneedle system

FIG. 3 shows a possible embodiment of the composite, comprising an oral thin film and an application aid for the oral thin film. The composite has a first area of the application aid on which the oral thin film is disposed, and a second area of the application aid which is designed in such a way that a user can touch the application aid in such a way that the oral thin film is not touched.

An adhesive layer (2) is applied to an application aid (1), to which the oral thin film (3) is applied in a first area. Optionally, a piece of the application aid (1a) can once again be applied to the adhesive layer in a second area, so that the user does not have to touch the adhesive layer.

The oral thin film is equipped with microneedles (5). These microneedles (5) can either be applied to the oral thin film with another adhesive layer (4) or attached thereon using alternative attachment measures.

FIG. 4 shows another possible embodiment of the composite, comprising an oral thin film and an application aid for the oral thin film. Here, the composite has a first area of the application aid on which the oral thin film is disposed, and a second area of the application aid which is designed in such a way that a user can touch the application aid in such a way that the oral thin film is not touched. The user is represented by the stylized hand (9).

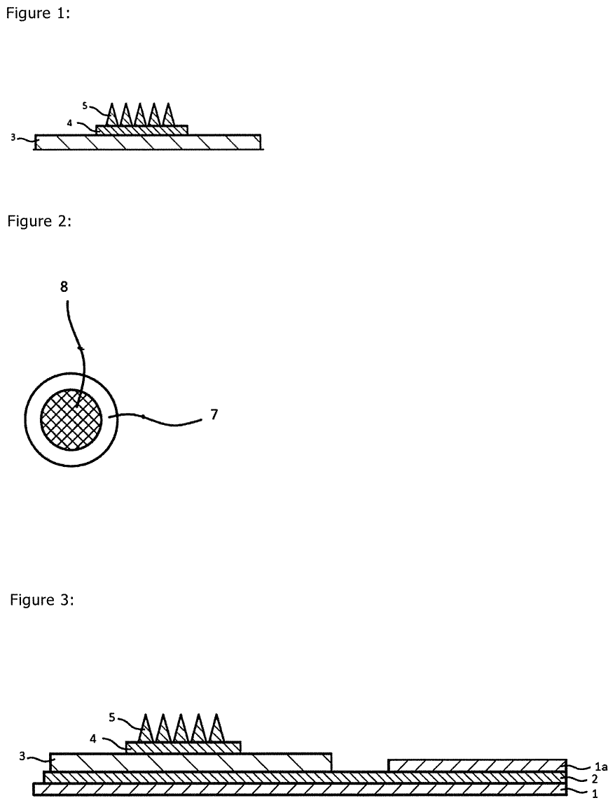

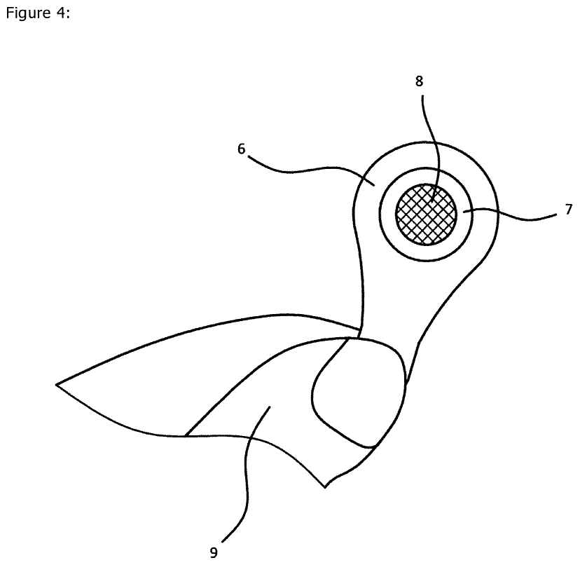

An oral thin film (7) is applied to an application aid (6) in an area that differs from the area that the user touches with his hand (9). The oral thin film (7) may be equipped with microneedles (8).

### EXAMPLES

The invention is explained below in more detail with reference to non-limiting examples.

### Example 1

An oral thin film was prepared using a microneedle system.

The microneedle system has a composition according to the following Table 1.

| Material | Content (% dry) | Description |
|---|---|---|
| Kollidon 30 ¹ | 97.50 | polymer |
| glycerol | 2.58 | plasticizer |
| trehalose | 6.07 | sugar substitute |
| Tween 80 | 0.3 | emulsifier |

¹ polyvinylpyrrolidone

All components are dissolved in the process solvent water. The mass is then dispensed onto silicone matrices (“negative mold”, specifies the number, shape, and length of the needles) and dried. The dried microneedle systems are demolded from the matrices and packaged.

The oral thin film has a composition according to the following Table 2.

TABLE 2

| Material | Content (% dry) | Description |
|---|---|---|
| Polyox WSR N10¹ | 97.50 | polymer |
| glycerol | 2.30 | plasticizer |
| FD&C Red No. 40² | 0.20 | dye |

¹polyethylene oxide with a molecular weight of about 100,000.
²dye

The raw materials were dissolved in water, coated onto a liner, and then dried in a drying oven. This resulted in a film with a weight per unit area of 96 g/m².

Two variants were selected for the application of the microneedles.

Both variants make use of the low melting point at which the Polyox in this formulation is in a soft, sticky state at the dry temperature of 70° C.

### Variant 1:

Immediately after the drying time has elapsed, the microneedles are placed with their back side onto the film, which is still at 70° C. Since the polyethylene oxide film is still soft, the microneedles will adhere. After cooling, the film solidifies and the microneedles are “welded”.

### Variant 2:

Microneedles are applied with their back onto a cold, solid polyethylene oxide film. The film is then heated to 70° C. to fix the microneedles. After cooling, they are then welded to the film.

### Example 2

Microneedles were attached onto an oral thin film by means of an adhesive layer. The microneedle system corresponds to that of example 1.

For this purpose, an adhesive layer with the composition shown in Table 3 was used.

TABLE 3

| Material | Content (% dry) | Description |
|---|---|---|
| Kollidon VA64¹ | 80 | polymer |
| glycerol | 20 | plasticizer |

¹polyvinylpyrrolidone-vinyl acetate copolymer

The raw materials were dissolved in ethanol, coated onto a siliconized liner, and then dried. The resulting adhesive layer had a weight per unit area of 47 g/m².

As the oral thin film, a soluble composition according to the following Table 4 was used.

TABLE 4

| Material | Content (% dry) | Description |
|---|---|---|
| Kollicoat MAE 100-55¹ | 20.95 | polymer |
| Kollicoat MAE100 P² | 62.95 | polymer |
| triethyl citrate | 10.00 | plasticizer |
| Cherry US | 3.00 | flavor |
| saccharin sodium | 2.00 | sweetener |
| sucralose | 1.00 | sweetener |
| menthol | 0.10 | flavor |

¹(meth)acrylic-ethyl acrylate copolymer
²(meth)acrylic-ethyl acrylate copolymer

The polymers were dissolved in a water/ethanol mixture, then the other raw materials were stirred in and homogenized. The mass was spread onto a siliconized liner and dried. This resulted in a film with a weight per unit area of 48 g/m²

The microneedles and the oral thin film were then joined together using the adhesive layer produced.

### Example 3

An oral thin film with microneedles was provided with a polyvinyl alcohol foam as an application aid. The microneedle system corresponds to that of example 1.

The application aid has the following formulation.

TABLE 5

| Material | Content %(dry) | Description |
|---|---|---|
| polyvinyl alcohol 4-88 | 84.8 | polymer |
| FD&C-Red no. 40 | 0.2 | dye |
| saccharin Na | 1.0 | sweetener |
| sucralose | 2.0 | sweetener |
| glycerol | 5.0 | plasticizer |
| cherry flavor | 6.0 | flavoring agent |
| menthol | 1.0 | flavoring agent |

A pre-solution of the polyvinyl alcohol was prepared in water. The other raw materials were then stirred into this pre-solution and dissolved. The foam used here was then foamed up using a foaming machine and dried on a pilot plant.

The oral thin film was applied to the application aid using an adhesive layer (analogous to example 2).

A stable composite is obtained. The very thin oral thin film, which tends to crease, could be applied. The presence of the microneedles in particular makes appropriate application necessary, since an inappropriate application will lead to the microneedles breaking off.

1. An oral thin film comprising a matrix layer containing at least one polymer, characterized in that a microneedle system is applied to at least one surface of the oral thin film, wherein the microneedle system is applied to the at least one surface of the oral thin film by means of an adhesive layer.

2. The oral thin film according to claim 1, characterized in that the at least one polymer comprises a water-soluble polymer, preferably selected from the group comprising starch, starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acid, polyacrylates, polyvinylpyrrolidones, vinylpyrrolidone-vinyl acetate copolymers, polyvinyl alcohols, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan, and/or natural gums.

3. The oral thin film according to claim 1, characterized in that the matrix layer and/or the microneedle system contain at least one active pharmaceutical ingredient.

4. The oral thin film according to claim 1, characterized in that the at least one active pharmaceutical ingredient is selected from the group comprising the active ingredient classes of analgesics, hormones, hypnotics, sedatives, antiepileptics, amphetamines, psychoneurotropic agents, neuromuscular-blocking agents, antispasmodics, antihistamines, antiallergics, cardiotonic agents, antiarrhythmics, diuretics, hypotensive agents, vasopressors, antidepressants, antitussives, expectorants, thyroid hormones, sex hormones, antidiabetics, antitumor agents, antibiotics, chemotherapeutics, and/or narcotics.

5. The oral thin film according to claim 1, characterized in that the oral thin film is present in the form of a solidified foam having cavities.

6. The oral thin film according to claim 1, characterized in that the microneedle system comprises microneedles having a length of 10 to 1000 μm.

7. The oral thin film according to claim 1, characterized in that the microneedle system is a microneedle system based on starch, starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acids, polyacrylates, polyvinylpyrrolidones, polyvinyl alcohols, poly(lactide-co-glycolide), hyaluronic acid, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan, and/or natural gums.

8. The oral thin film according to claim 1, characterized in that the oral thin film and/or the microneedle system further comprise at least one excipient selected from the group comprising colorants, flavors, sweeteners, plasticizers, taste-masking agents, emulsifiers, enhancers, pH regulators, humectants, preservatives, and/or antioxidants.

9. A composite comprising an oral thin film according to claim 1 and an application aid, which comprises at least one polymer, for the oral thin film, characterized in that the oral thin film and the application aid are firmly joined to each other.

10. The composite according to claim 9, characterized in that the application aid has a surface area which is at least twice as large as the surface area of the oral thin film.

11. The composite according to claim 9, characterized in that the application aid has a surface area of 1 cm² to 30 cm².

12. The composite according to claim 9, characterized in that the application aid comprises at least one water-soluble polymer.

13. A method of preparing an oral thin film according to claim 1, comprising the steps of
providing an oral thin film comprising at least one polymer and at least one active pharmaceutical ingredient,
providing a microneedle system, and
firmly joining the microneedle system and the oral thin film, wherein firmly joining comprises the following steps
applying an adhesive layer to a surface of the oral thin film,
applying the microneedle system to this adhesive layer, and
drying the adhesive layer to obtain a firm composite.

14. A method for preparing a composite comprising an oral thin film and an application aid for the oral thin film according to claim 9, comprising the steps of:
providing an application aid,
providing an oral thin film, and
firmly joining the application aid to the oral thin film.

15. The method according to claim 14, characterized in that firmly joining the oral thin film and the application aid comprises the following steps
heating the oral thin film and/or the application aid to a temperature that is above the softening temperature of the respective at least one polymer,
applying the oral thin film to a surface of the application aid to obtain a loose composite, and
cooling the loose composite to obtain a firm composite.

16. The method according to claim 14, characterized in that firmly joining of the application aid and the oral thin film comprises the following steps
applying an adhesive layer to a surface of the oral thin film or to a surface of the application aid,
joining the application aid and the oral thin film via this adhesive layer, and
drying the adhesive layer to obtain a firm composite.

17. (canceled)

18. (canceled)

19. A method for administering a medicinal product comprising the providing of the oral thin film of claim 1 to a patient.

20. The oral thin film according to claim 1, characterized in that the microneedle system comprises microneedles having a length of 100 to 600 μm.

21. The oral thin film according to claim 1, characterized in that the microneedle system comprises microneedles having a length of 250 μm to 350 μm.

22. The composite according to claim 9, characterized in that the application aid has a surface area which is 2 to 10 times as large as the surface area of the oral thin film.
